(19) United States
(12) Patent Application Publication
(10) Pub. No.: US 2025/0136736 A1
(43) Pub. Date: May 1, 2025

(54) PREPARATION OF TERTIARY ESTER-CONTAINING AROMATIC VINYL MONOMER

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 18/918,334

(22) Filed: Oct. 17, 2024

(30) Foreign Application Priority Data
Oct. 30, 2023 (JP) 2023-185147

(51) Int. Cl. C08F 220/60; C08F 216/08; C08F 216/10; C08F 220/12; C08F 220/18; C08F 220/40

(52) U.S. Cl. CPC C08F 220/60; C08F 216/08; C08F 216/10; C08F 220/12; C08F 220/1803; C08F 220/40

(57) ABSTRACT

A tertiary ester-containing aromatic vinyl monomer is prepared by reacting an N-acylimidazole compound with a tertiary alcohol compound in the presence of a metal alkoxide as a reaction promoter. The tertiary ester-containing aromatic vinyl monomer of quality is prepared at a high efficiency and is applicable to resist compositions adapted for the EB and EUV lithography processes.

### CROSS-REFERENCE TO RELATED APPLICATIONS

This non-provisional application claims priority under 35 U.S.C. § 119(a) on Patent Application No. 2023-185147 filed in Japan on Oct. 30, 2023, the entire contents of which are hereby incorporated by reference.

### TECHNICAL FIELD

This invention relates to a method for preparing a tertiary ester-containing aromatic vinyl monomer.

### BACKGROUND ART

To meet the demand for higher integration density of LSIs, the effort to reduce the pattern rule is in rapid progress. Acid-catalyzed chemically amplified resist compositions are most often used in forming resist patterns with a feature size of 0.2 μm or less. High-energy radiation such as UV, deep-UV or EB is used as the light source for exposure of these resist compositions. In particular, the EB lithography, which is utilized as the ultra-fine microfabrication technique, is also indispensable in processing photomask blanks to form photomasks for use in semiconductor device fabrication.

Polymers comprising a major proportion of aromatic structure having an acidic side chain, for example, polyhydroxystyrene are useful in resist materials for the KrF excimer laser lithography. These polymers are not used in resist materials for the ArF excimer laser lithography because they exhibit strong absorption to radiation of wavelength around 200 nm. These polymers, however, are expected to form useful resist materials for the EB and EUV lithography for forming patterns of smaller size than the processing limit of ArF excimer laser because they offer high etching resistance.

Positive resist compositions adapted for the EB and EUV lithography typically use a base polymer having on a phenol side chain an acidic functional group which is masked with an acid decomposable protective group (or acid labile group). The photoacid generator generates an acid upon exposure to high-energy radiation. The generated acid serves as a catalyst to deprotect the protective group so that the base polymer may be solubilized in an alkaline developer. Typical of the acid decomposable protective group are tertiary alkyl groups, tert-butoxycarbonyl groups and acetal groups. On use of protective groups requiring a relatively low level of activation energy for deprotection such as acetal groups, a resist film having a high sensitivity is advantageously obtainable. However, if the diffusion of generated acid is not fully controlled, deprotection reaction can occur even in the unexposed regions of the resist film, giving rise to problems like degradation of line edge roughness (LER) and a lowering of critical dimension uniformity (CDU) of patterns.

It is pointed out that a resist composition adapted for the ArF lithography comprising a polymer having a carboxy group (of methacrylic acid or the like) substituted with an acid labile group is swollen in alkaline developer. By contrast, the resist composition adapted for the KrF lithography comprising a polymer having a phenol group (of hydroxystyrene or the like) substituted with an acid labile group experiences a less amount of swell. The hydroxystyrene, however, allows for significant acid diffusion, leaving the risk of resolution lowering. Also, structural units of styrenecarboxylic acid having a carboxyl group substituted with an acid labile group are proposed in Patent Documents 1 to 3.

Patent Documents 1 to 3 disclose the method of preparing a tertiary ester-containing vinyl aromatic monomer by converting the carboxy group of styrenecarboxylic acid or vinylnaphthalenecarboxylic acid to a tertiary ester. Specifically, one disclosed method involves deriving styrenecarboxylic acid to a corresponding acid chloride using a corresponding tertiary alcohol, and effecting esterification reaction in the presence of an organic base such as triethylamine. Another method involves the steps of letting an organometallic reagent act on a ketone compound to generate a tertiary alkoxide, reacting it with styrenecarboxylic chloride to form a tertiary ester-containing styrene monomer. However, several problems remain unsolved with respect to the conversion rate of reaction and competing anionic polymerization during reaction.

To meet the future requirement of resist patterns to smaller size, an improvement in the preparation of tertiary ester-containing vinyl aromatic monomers is important. It is desired to develop an efficient method of preparing a tertiary ester-containing vinyl aromatic monomer of quality.

### CITATION LIST

Patent Document 1: JP-A 2006-335715

Patent Document 2: JP 7055070

Patent Document 3: JP 6694451

### SUMMARY OF THE INVENTION

In conjunction with acid-catalyzed chemically amplified resist compositions, a tertiary ester-containing vinyl aromatic monomer is regarded important for forming patterns having a small feature size. It is desired to develop a method of preparing a tertiary ester-containing vinyl aromatic monomer, the method being more efficient and the monomer being of higher quality than in the prior art formulation.

An object of the invention is to provide an efficient method of preparing a tertiary ester-containing vinyl aromatic monomer of quality which is applicable in resist compositions adapted for the EB lithography and EUV (wavelength 13.5 nm) lithography.

The inventor has found that when a tertiary ester-containing vinyl aromatic monomer is prepared using a metal alkoxide as a reaction promoter, the monomer of higher quality is obtained in higher yields than in the prior art methods.

In one aspect, the invention provides a method for preparing a tertiary ester-containing aromatic vinyl monomer wherein a metal alkoxide is used as a reaction promoter.

The method typically involves the step of reacting an N-acylimidazole compound with a tertiary alcohol compound using a metal alkoxide as a reaction promoter.

In the preferred embodiment, the N-acylimidazole compound has the formula (A1), the tertiary alcohol compound has the formula (A2), and the tertiary ester-containing aromatic vinyl monomer has the formula (A3).

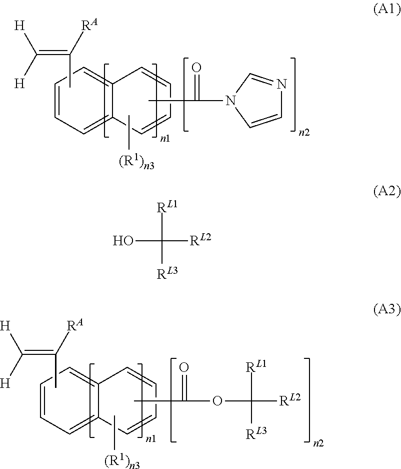

Herein n1 is 0 or 1, n2 is 1 or 2, n3 is an integer of 0 to 6, n2+n3 is from 1 to 5 when n1=0 and n2+n3 is from 1 to 7 when n1=1. R^{A} is hydrogen, fluorine, methyl or trifluoromethyl. R¹ is halogen, a C₁-C₂₀ hydrocarbyl group which may contain a heteroatom, or C₁-C₂₀ hydrocarbyloxy group which may contain a heteroatom. R^{L1}, R^{L2} and R^{L3} are each independently a C₁-C₃₀ hydrocarbyl group, any two of R^{L1}, R^{L2} and R^{L3} may bond together to form a ring with the carbon atom to which they are attached, and —CH₂— in the hydrocarbyl group and the ring may be replaced by —O— or —S—.

The metal alkoxide is preferably an alkali metal alkoxide or alkaline earth metal alkoxide, more preferably an alkali metal alkoxide.

The alkali metal alkoxide preferably has a sodium or potassium cation. Also preferably, the alkali metal alkoxide has an alkoxide anion containing a tertiary carbon atom.

### ADVANTAGEOUS EFFECTS OF INVENTION

The preparation method of the invention ensures that a tertiary ester-containing aromatic vinyl monomer of high quality is obtained in high yields.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the asterisk (*) designate a valence bond or point of attachment. The terms “group” and “moiety” are interchangeable. EB stands for electron beam and EUV for extreme ultraviolet.

One embodiment of the invention is a method for preparing a tertiary ester-containing aromatic vinyl monomer wherein a metal alkoxide is used as a reaction promoter. Specifically, a tertiary ester-containing aromatic vinyl monomer is prepared by reacting an N-acylimidazole compound with a tertiary alcohol compound using a metal alkoxide as a reaction promoter.

In a preferred embodiment, the N-acylimidazole compound has the formula (A1), which is also referred to as compound A1; the tertiary alcohol compound has the formula (A2), which is also referred to as compound A2; and the tertiary ester-containing aromatic vinyl monomer has the formula (A3), which is also referred to as compound A3.

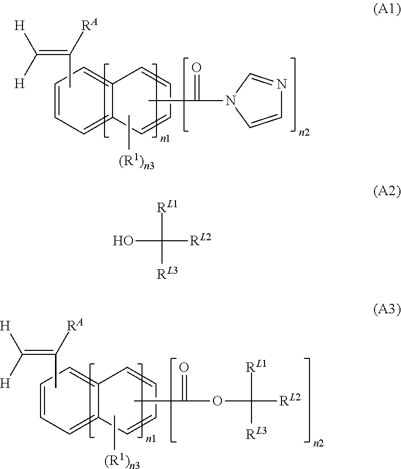

In formulae (A1) and (A3), n1 is 0 or 1. The relevant structure is a benzene ring when n1=0 and a naphthalene ring when n1=1. A benzene ring corresponding to n1=0 is more preferred from the aspect of solvent solubility. The subscript n2 is 1 or 2, and n3 is an integer of 0 to 6, with the proviso that n2+n3 is from 1 to 5 when n1=0 and n2+n3 is from 1 to 7 when n1=1.

In formulae (A1) and (A3), R^{A} is hydrogen, fluorine, methyl or trifluoromethyl. Inter alia, hydrogen or methyl is preferred, with hydrogen being more preferred.

In formulae (A1) and (A3), R¹ is halogen, a C₁-C₂₀ hydrocarbyl group which may contain a heteroatom, or a C₁-C₂₀ hydrocarbyloxy group which may contain a heteroatom.

Suitable halogen atoms include fluorine, chlorine, bromine and iodine, with fluorine and iodine being preferred.

The hydrocarbyl group and hydrocarbyl moiety in the hydrocarbyloxy group may be saturated or unsaturated and straight, branched or cyclic. Examples thereof include C₁-C₂₀ alkyl groups such as methyl, ethyl, n-propyl, isopropyl, n-butyl, and tert-butyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, allyl, butenyl and hexenyl; C₃-C₂₀ cyclic unsaturated hydrocarbyl groups such as cyclohexenyl; C₆-C₂₀ aryl groups such as phenyl and naphthyl; C₇-C₂₀ aralkyl groups such as benzyl, 1-phenylethyl and 2-phenylethyl; and combinations thereof. Inter alia, aryl groups are preferred. In the hydrocarbyl group, some or all of the hydrogen atoms may be substituted by a moiety containing a heteroatom such as oxygen, sulfur, nitrogen or halogen, and some constituent—CH₂— may be replaced by a moiety containing a heteroatom such as oxygen, sulfur or nitrogen, so that the group may contain a hydroxy, cyano, fluorine, chlorine, bromine, iodine, carbonyl, ether bond, ester bond, sulfonate ester bond, carbonate bond, lactone ring, sultone ring, carboxylic anhydride (—C(═O)—O—C(═O)—) or haloalkyl moiety. When n3 is 2 or more, a plurality of R¹ may bond together to form a ring with the carbon atoms on the aromatic ring to which they are attached.

The compound of N-acylimidazole structure having formula (A1) is preferably obtained by mixing a corresponding aromatic carboxylic acid and N,N-carbonyldiimidazole. After the compound is formed in the reaction system, it may be used in the subsequent reaction without further purification.

The aromatic carboxylic acid which is a precursor for the compound of N-acylimidazole structure having formula (A1) is preferably an optionally substituted styrenecarboxylic acid or vinylnaphthalene carboxylic acid. It may be a commercially available one or furnished through well-known organic synthesis reaction.

In formulae (A2) and (A3), R^{L1}, R^{L2} and R^{L3} are each independently a C₁-C₃₀ hydrocarbyl group. Any two of R^{L1}, R^{L2} and R^{L3} may bond together to form a ring with the carbon atom to which they are attached. Also, —CH₂— in the hydrocarbyl group and the ring may be replaced by —O— or —S—.

Examples of the structure (acid labile group): —C(R^{L1})(R^{L2})(R^{L3}) in formulae (A2) and (A3) are shown below, but not limited thereto. It is noted that the asterisk (*) designates a point of attachment to the adjoining hydroxy group.

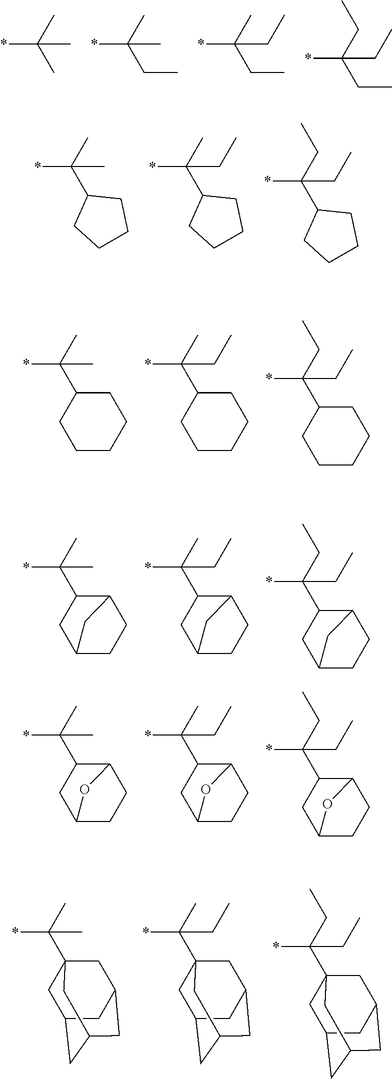

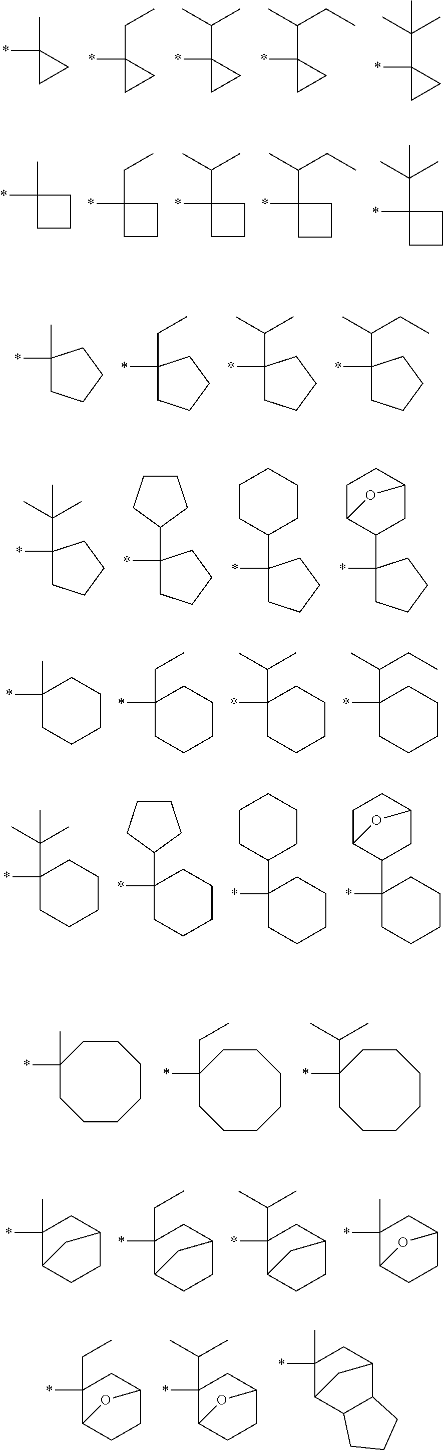

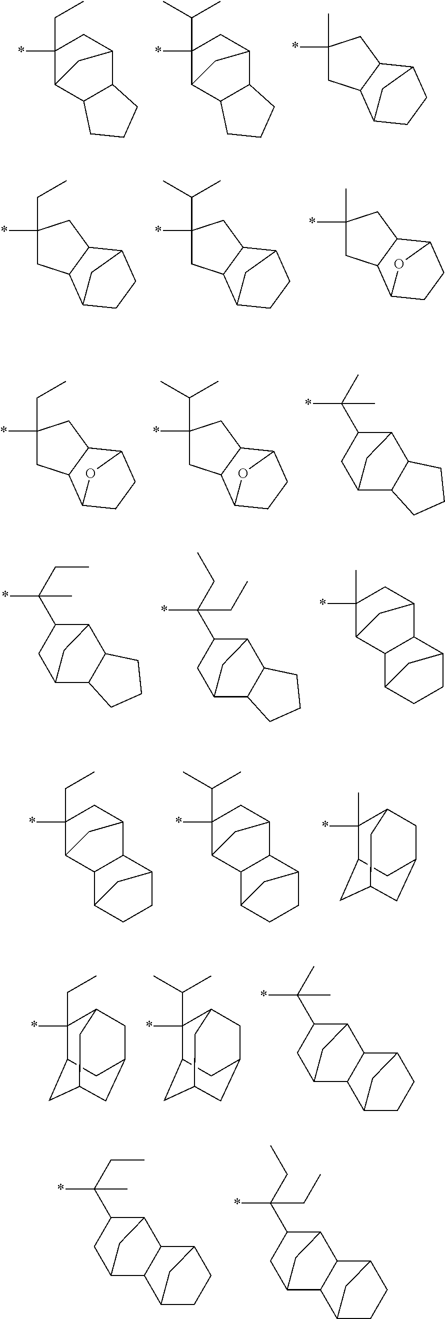

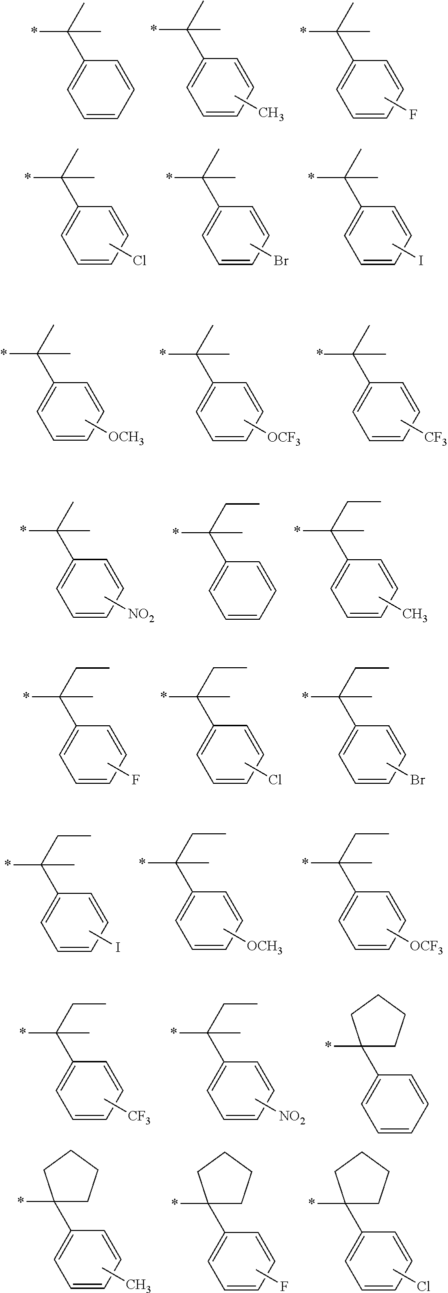

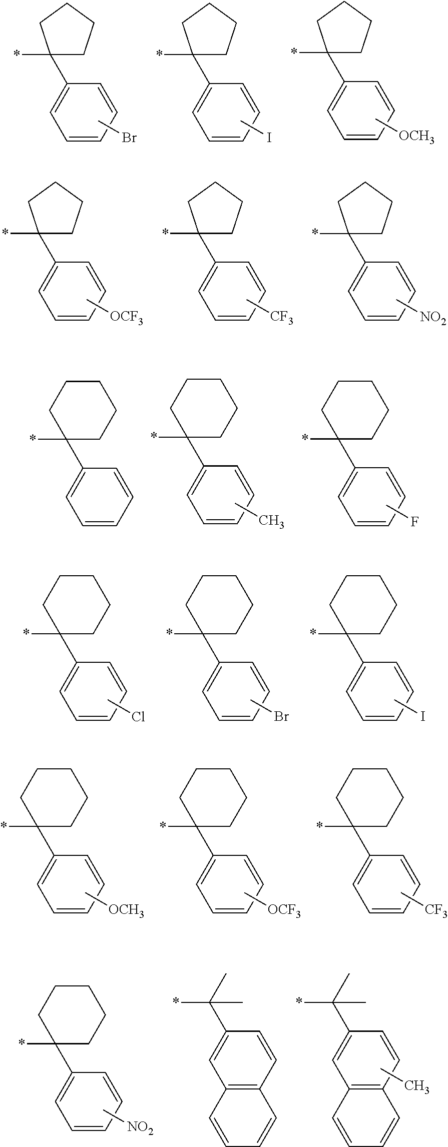

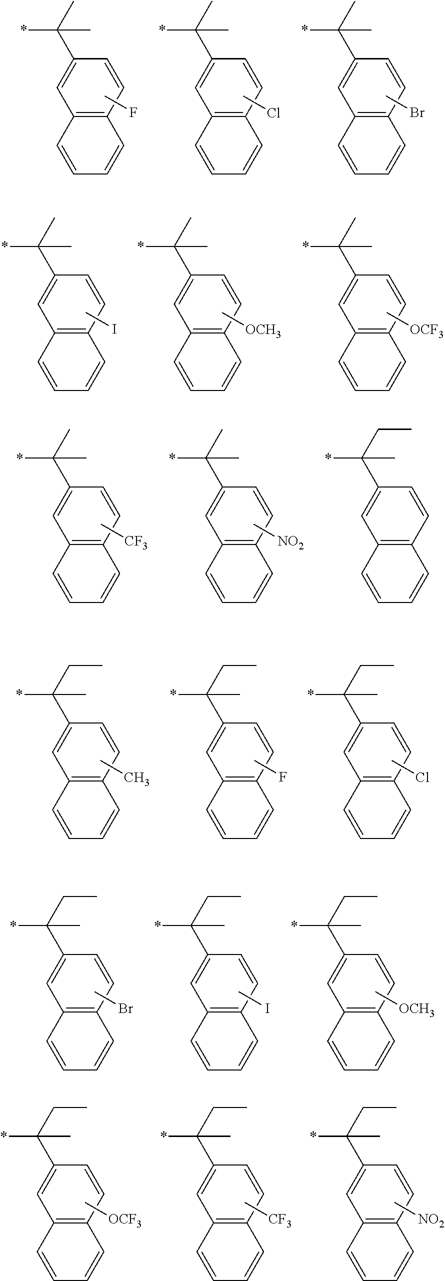

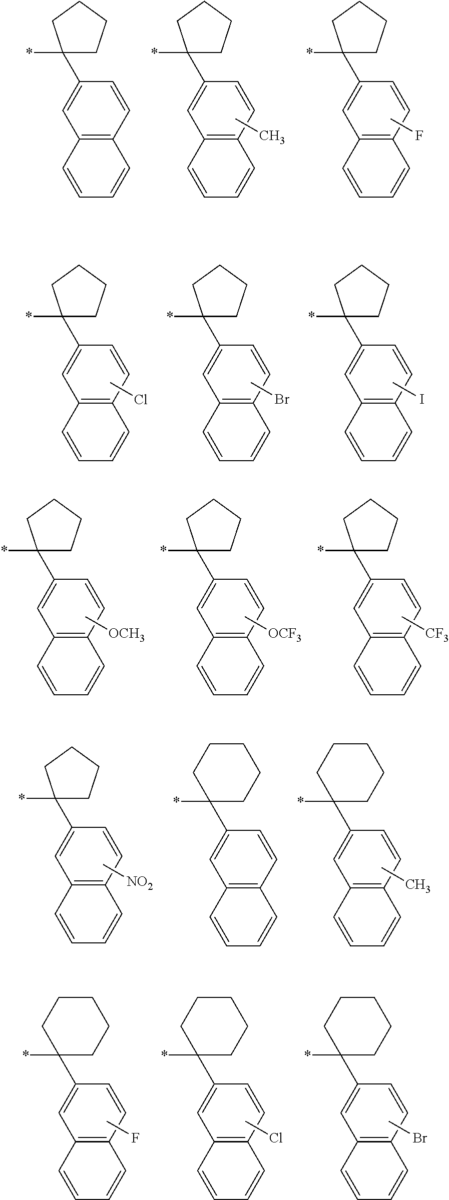

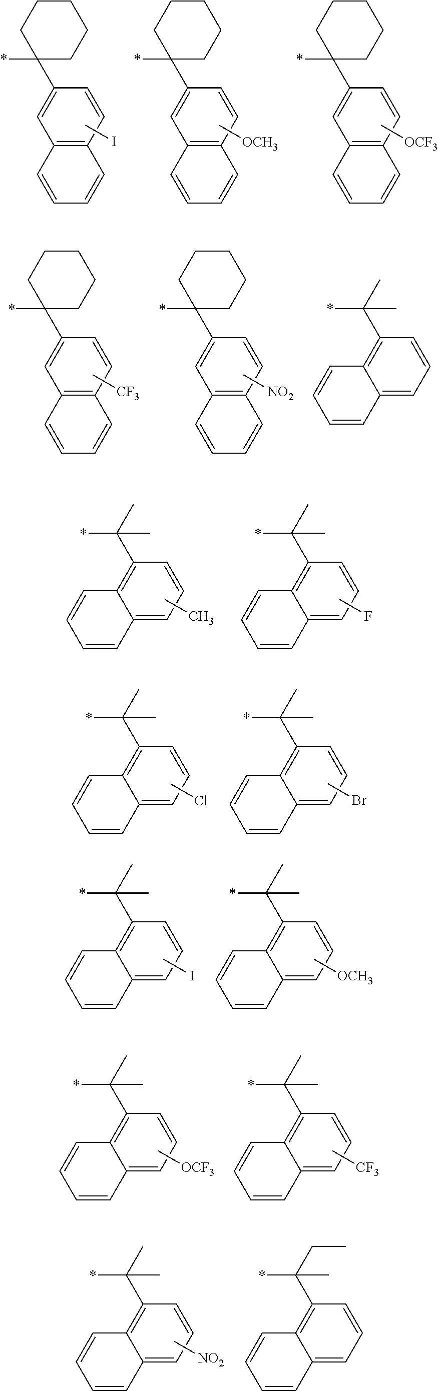

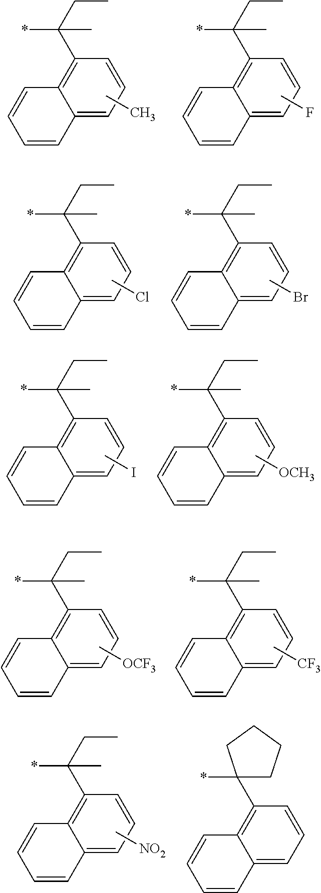

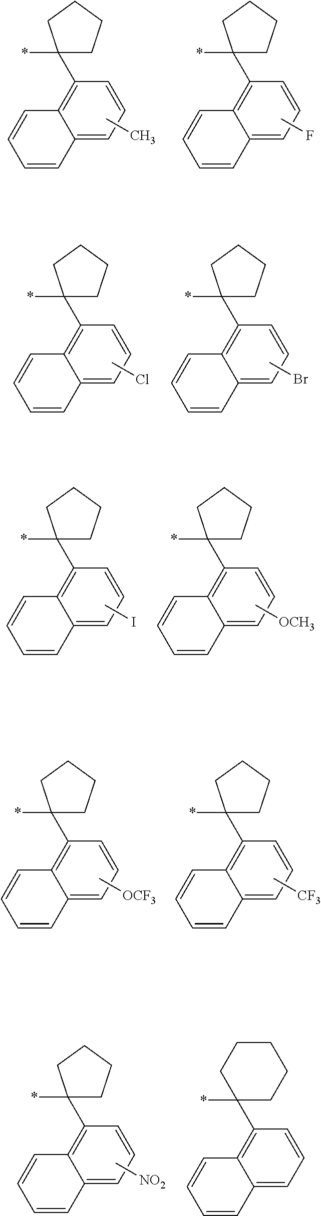

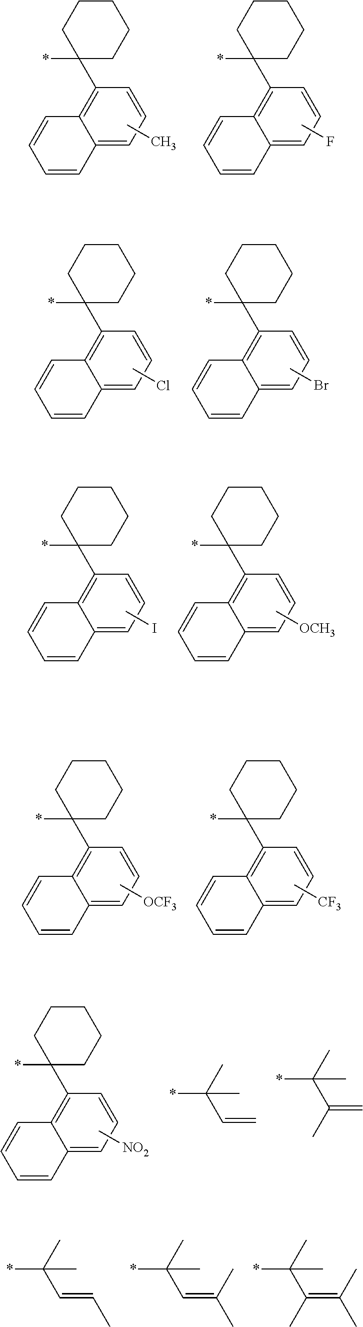

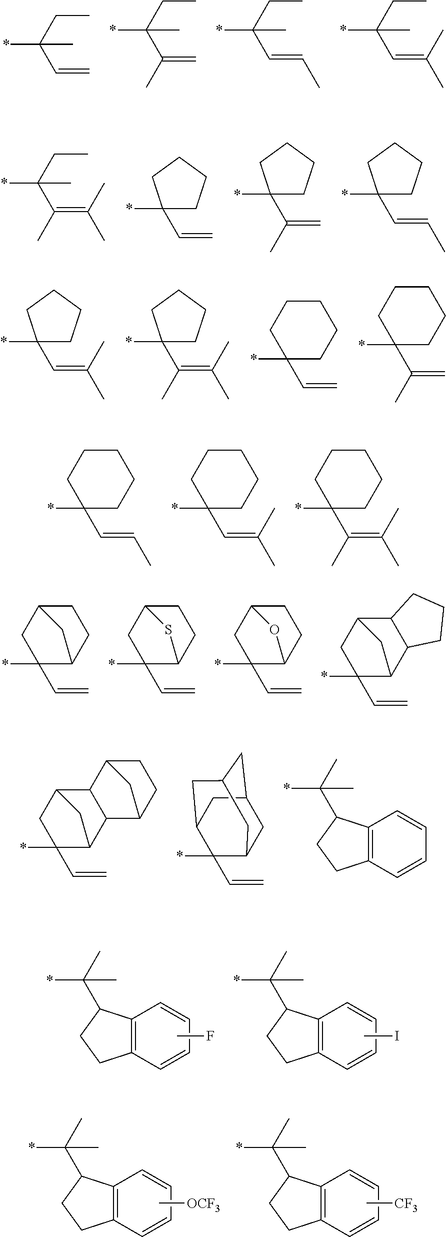

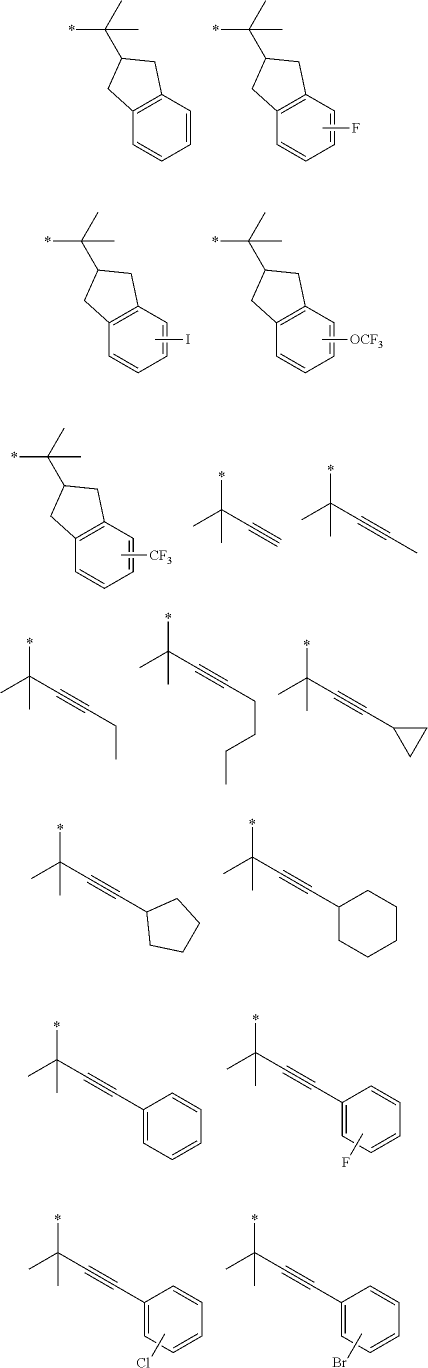

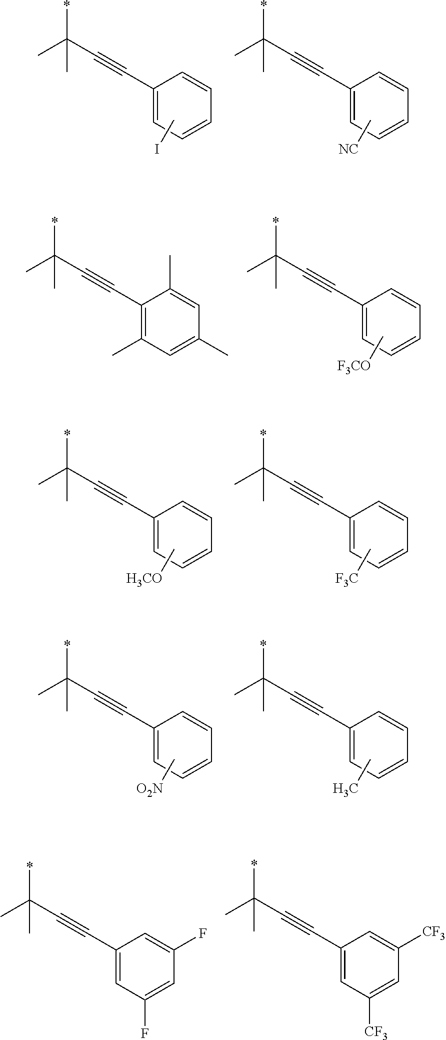

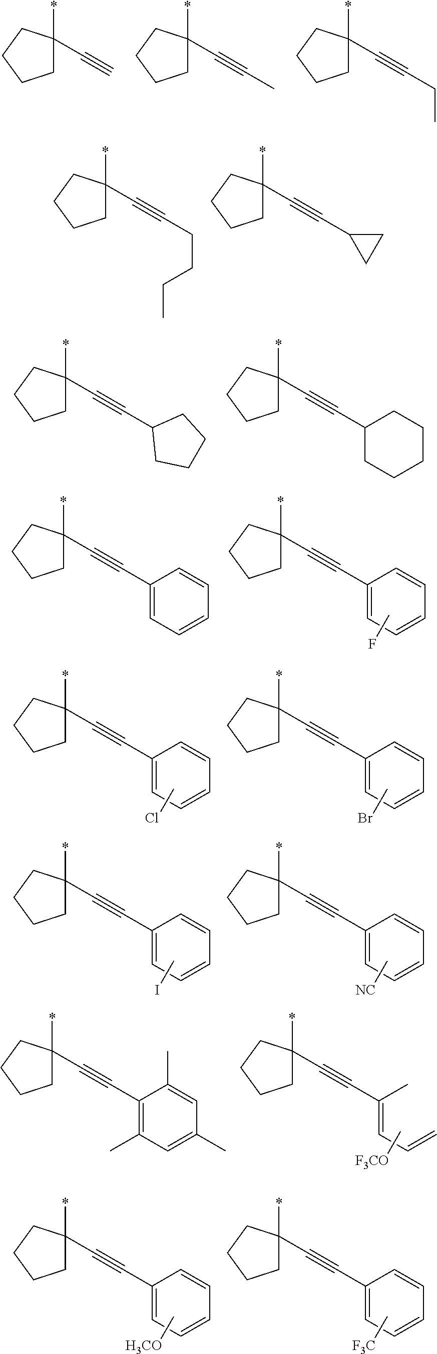

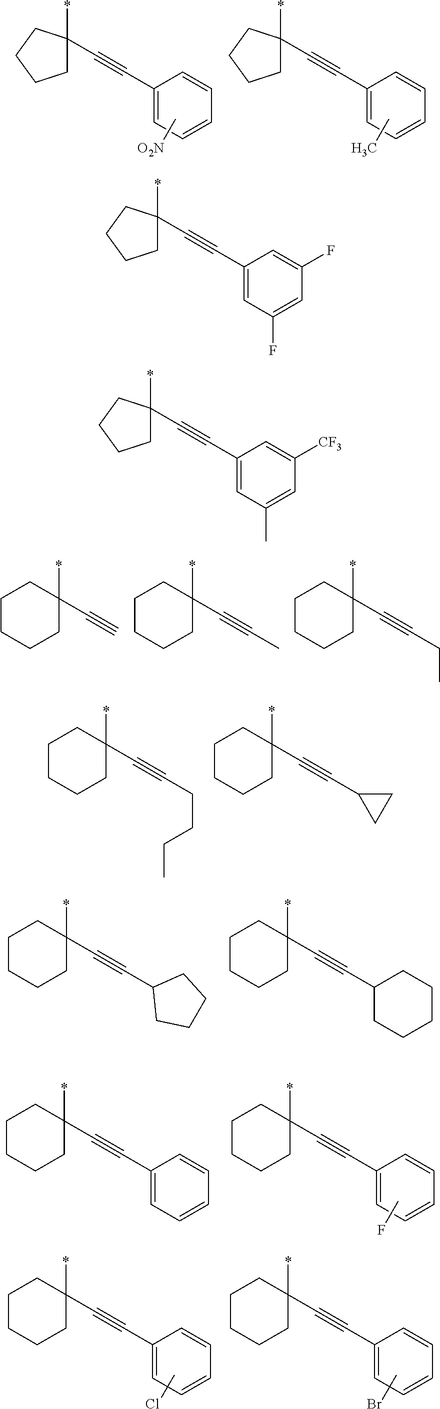

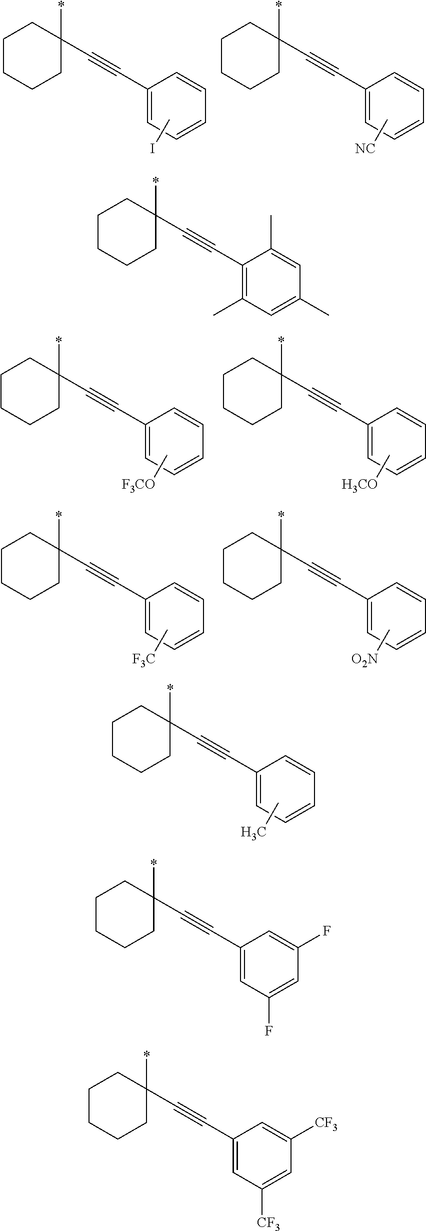

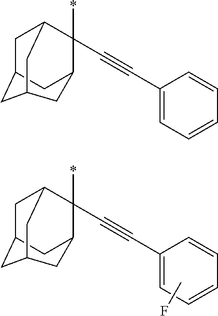

In the reaction of compound A1 with compound A2, the amount of compound A2 used is not particularly limited. From the aspect of obtaining compound A3 in high yields, the amount of compound A2 used is preferably 1.0 to 2.0 moles, more preferably 1.0 to 1.5 moles per mole of compound A1.

In the reaction of compound A1 with compound A2, the metal alkoxide is used as the reaction promoter in order to form compound A3 in high yields. The metal alkoxide is preferably an alkali metal alkoxide or alkaline earth metal alkoxide.

Examples of the alkali metal alkoxide include lithium alkoxides, sodium alkoxides and potassium alkoxides. Of these, sodium alkoxides and potassium alkoxides are preferred from the aspects of availability and preparation. Examples of the alkaline earth metal alkoxide include magnesium alkoxides, calcium alkoxides, and strontium alkoxides. Of these, magnesium alkoxides are preferred from the aspects of availability and preparation.

The preferred metal alkoxide is an alkali metal alkoxide.

The metal alkoxide has an anion, examples of which include alkoxide anions containing primary carbon, alkoxide anions containing secondary carbon, and alkoxide anions containing tertiary carbon. Inter alia, the alkoxide anions containing tertiary carbon are preferred.

Examples of the alkoxide anion containing tertiary carbon include t-butoxy,

t-amyloxy and 1-adamantyloxy anions, with the t-butoxy anion being preferred from the aspect of availability. While an alkoxide anion is obtained from deprotonation of the hydroxy group in compound A2, it is acceptable to separately prepare such an alkoxide anion and use it in the reaction.

The alkoxide anion obtained from deprotonation of the hydroxy group in compound A2 may be prepared by letting an alkali metal hydride such as sodium hydride or potassium hydride or an organometallic reagent such as n-butyl lithium or Grignard reagent act on compound A2 although the preparation method is not limited thereto.

The metal alkoxide is preferably used in an amount of 0.01 to 0.4 mole, more preferably 0.01 to 0.2 mole per mole of compound A1.

For the reaction of compound A1 with compound A2, a solvent may be used. The solvent used herein is not particularly limited as long as it is inert to the reaction. Suitable solvents include aromatic hydrocarbon solvents such as toluene and xylene; aliphatic hydrocarbon solvents such as hexane, heptane, octane and cyclohexane; ether solvents such as diethyl ether, diisopropyl ether, t-butyl methyl ether, tetrahydrofuran, and dioxane; polar aprotic solvents such as acetonitrile, N,N-dimethylformamide and N,N-dimethylacetamide. The solvents may be used alone or in admixture. Although the amount of the solvent used may be selected as appropriate, the amount is typically about 100 to about 500 parts by weight per 100 parts by weight of compound A1.

In the reaction of compound A1 with compound A2, a polymerization inhibitor may be added for inhibiting polymerization in the reaction system. Non-limiting examples of the polymerization inhibitor include hydroquinone monomethyl ether, hydroquinone, 4-hydroxy-2,2,6,6-tetramethylpiperidine-N-oxyl, 4-t-butylpyrocatechol, t-butylhydroquinone, and dibutylhydroxytoluene, phenothiazine, and copper salts. The inhibitor may be used alone or in admixture.

In the reaction of compound A1 with compound A2, the reaction temperature may fall in the range for ordinary esterification reaction. The reaction temperature is preferably from 40° C. to the boiling point of the solvent used from the aspect of reducing the reaction time, more preferably up to 100° C. for inhibiting side reactions or polymerization reaction, even more preferably up to 80° C. Since the reaction time varies with the reaction temperature and other conditions, it may be determined as appropriate. The reaction time is preferably about 0.5 to 30 hours.

The compound A3 resulting from the reaction of compound A1 with compound A2 is preferably purified. For the purification, any of well-known purifying techniques including alkaline water washing, water washing, distillation, recrystallization and filtration may be selected in combination while taking into account the physical properties of the product and the type of the solvent.

### EXAMPLES

Examples of the invention are given below by way of illustration and not by way of limitation. Analysis is made by IR and NMR spectroscopy using analytic instruments as shown below.
IR: NICOLET 6700 by Thermo Fisher Scientific Inc.
¹H-NMR: ECA-500 by JEOL Ltd.

### [1] Synthesis of Monomers

### [Example 1] Route 1 for the Synthesis of Monomer A3-1

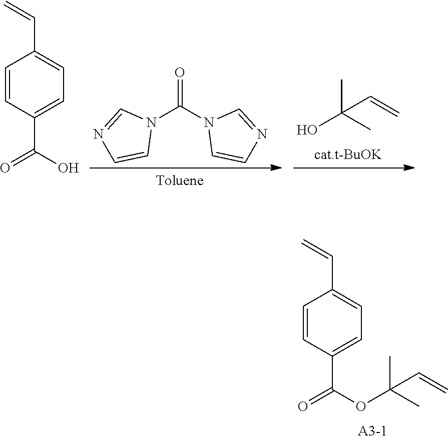

In a reactor under nitrogen atmosphere, 102.2 g of N,N-carbonyldiimidazole was suspended in 270 g of toluene. To the suspension was added 88.9 g of 4-styrenecarboxylic acid. Bubbling was observed. After it was confirmed that the reaction system turned transparent, the reactor was heated at 50° C., followed by 2 hours of aging. At the end of aging, 3.37 g of t-butoxypotassium was added, and 62.0 g of 2-methyl-3-buten-2-ol was then added dropwise. At the end of addition, the reactor was heated at 80° C., followed by 24 hours of aging. After the reaction solution was cooled, 150 g of 1 wt % sodium hydroxide aqueous solution was added thereto to quench the reaction. Ordinary aqueous workup was then performed, the solvent was distilled off, and the product was purified by distillation. Monomer A3-1 was obtained as colorless oily matter. Amount 122.0 g, yield 94%.

The IR spectral data and ¹H-NMR of Monomer A3-1 are shown below.

IR (D-ATR): v=3089, 2981, 2936, 1714, 1644, 1630, 1608, 1567, 1507, 1470, 1403, 1379, 1365, 1312, 1285, 1236, 1201, 1178, 1166, 1136, 1105, 1016, 989, 917, 861, 783, 713, 683, 433 cm⁻¹

¹H-NMR (600 MHz in DMSO-d6): δ=7.87 (2H, d), 7.58 (2H, d), 6.80 (1H, dd), 6.17 (1H, dd), 5.96 (1H, d), 5.40 (1H, d), 5.25 (1H, d), 5.10 (1H, d), 1.59 (6H, s) ppm

### Examples 2 to 10

Monomers having the structure shown in Tables 1 and 2 were synthesized according to the formulation of Example 1 using the corresponding reactants. It is noted that each of N-acylimidazole intermediates in the column of A1 in Tables 1 and 2 was synthesized from a corresponding aromatic carboxylic acid and N,N-carbonyl diimidazole and used in the subsequent step without further purification. The starting compounds A1 and A2 and the resulting monomers A3 have the structure shown in Tables 1 and 2.

TABLE 1

| | | | | | Isolation |
|---|---|---|---|---|---|
| | A1 | A2 | A3 | Metal alkoxide | yield (%) |
| Example 1 | 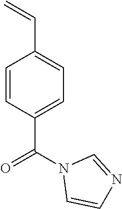 | 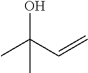 | 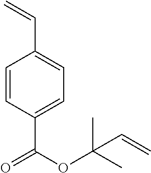 | t-BuOK | 94 |
| Example 2 | 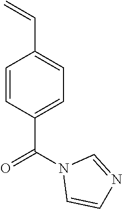 | 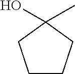 | 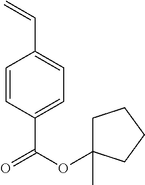 | t-BuOK | 90 |
| Example 3 | 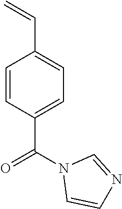 | 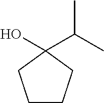 | 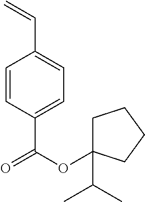 | t-BuOK | 89 |
| Example 4 | 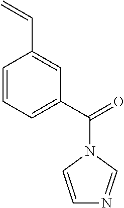 | 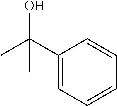 | 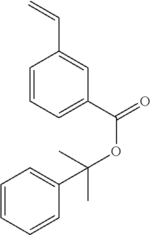 | t-BuOK | 90 |
| Example 5 | 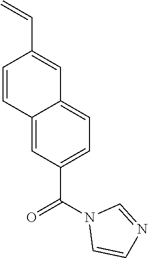 | 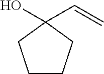 | 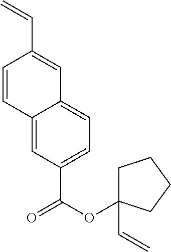 | t-BuONa | 91 |

TABLE 2

| | | | | | Isolation |
|---|---|---|---|---|---|
| | A1 | A2 | A3 | Metal alkoxide | yield (%) |
| Example 6 | 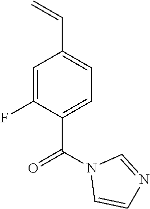 | 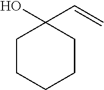 | 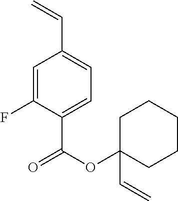 | MeONa | 86 |
| Example 7 | 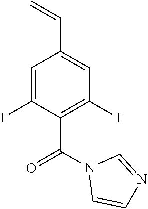 | 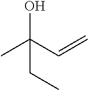 | 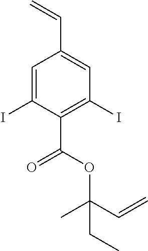 | 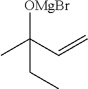 | 87 |
| Example 8 | 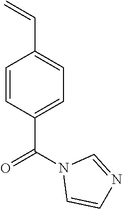 | 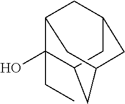 | 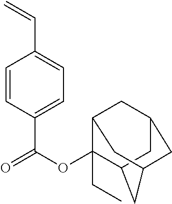 | 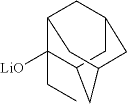 | 85 |
| Example 9 | 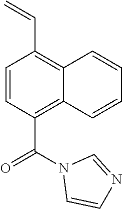 | 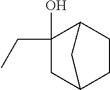 | 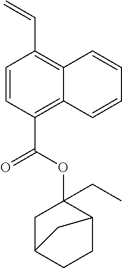 | 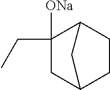 | 86 |
| Example 10 | 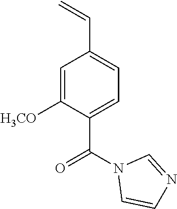 | 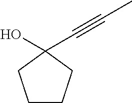 | 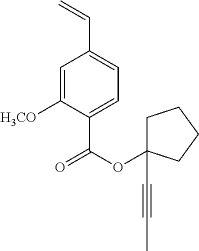 | t-BuOLi | 85 |

### [Comparative Example 1] Route 2 for the Synthesis of Monomer A3-1

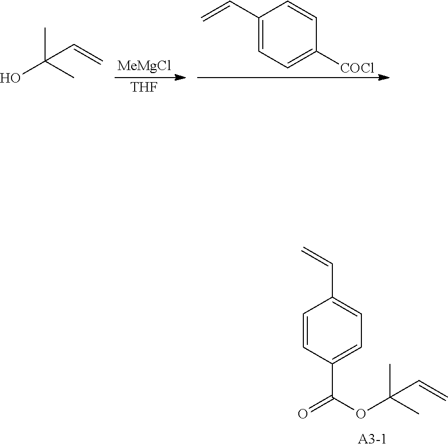

Under nitrogen atmosphere, 51.7 g of 2-methyl-3-buten-2-ol in 100 mL of THF was added dropwise to 200 mL of a 3.0 mol/L solution of methylmagnesium chloride in THF while cooling in an ice bath. This was followed by 1 hour of stirring. After a solution was obtained by preparing an acid chloride from 106.7 g of p-styrenecarboxylic acid and dissolving the acid chloride in 100 mL of THF, it was added dropwise to the reaction solution while cooling in an ice bath. This was followed by 3 hours of stirring at room temperature. The reaction solution was cooled, whereupon 250 g of a saturated sodium bicarbonate aqueous solution was added to quench the reaction. Since insolubles precipitated in the reaction solution, they were removed by filtration. 400 mL of toluene was added to the filtrate to extract the target compound. Ordinary aqueous workup was then performed, the solvent was distilled off, and the product was purified by silica gel column chromatography. Monomer A3-1 was obtained as colorless oily matter. Amount 38.9 g, yield 30%.

### [Comparative Example 2] Route 3 for the Synthesis of Monomer A3-1

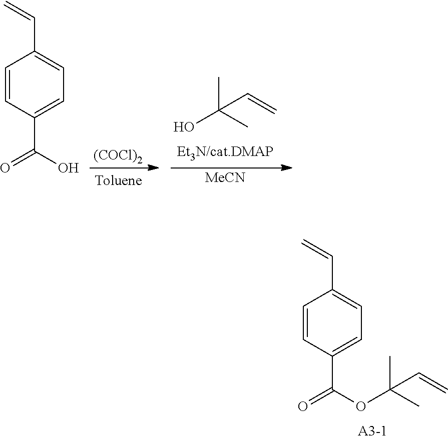

Under nitrogen atmosphere, 106.7 g of p-styrenecarboxylic acid was suspended in 500 mL of toluene. 109.7 g of oxalyl chloride was added dropwise to the suspension. At the end of addition, the reactor was heated at an internal temperature of 50° C., after which the reaction solution was aged for 6 hours. The reaction solution was then cooled to room temperature and concentrated, obtaining an acid chloride. To the concentrate were added 51.7 g of 2-methyl-3-buten-2-ol, 7.3 g of 4-dimethylaminopyridine, and 450 mL of acetonitrile. The solution was cooled in an ice bath. A mixture of 85.0 g of triethylamine and 50 mL of acetonitrile was added dropwise to the solution while maintaining the temperature of the reactor below 20° C. At the end of addition, the reactor was heated at a temperature of 60° C., followed by 18 hours of aging. The reaction solution was cooled, and 250 g of a saturated sodium bicarbonate aqueous solution was added to quench the reaction. This was followed by extraction with 400 mL of toluene, ordinary aqueous workup, and solvent distillation. The product was purified by silica gel column chromatography. Monomer A3-1 was obtained as colorless oily matter. Amount 22.1 g, yield 17%.

### [Comparative Example 3] Route 4 for the Synthesis of Monomer A3-1

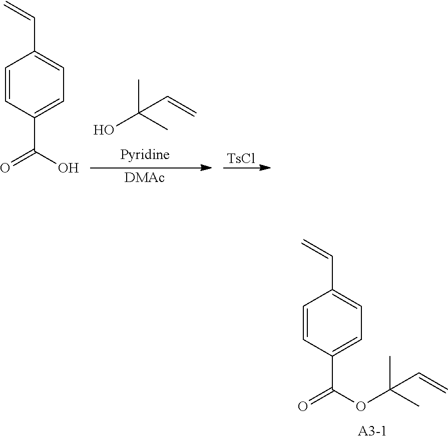

Under nitrogen atmosphere, 88.9 g of p-styrenecarboxylic acid, 189.8 g of pyridine, and 62.0 g of 2-methyl-3-buten-2-ol were dissolved in 216 g of N,N-dimethylacetamide. The reactor was heated at an internal temperature of 50° C., a solution of 137.3 g of p-toluenesulfonyl chloride and 120 g of N,N-dimethylacetamide was added dropwise. The reactor was heated at a temperature of 70° C., followed by 18 hours of aging. The reaction solution was cooled, whereupon 360 g of a 5% sodium hydroxide aqueous solution was added to quench the reaction. This was followed by extraction with 600 mL of toluene, ordinary aqueous workup, and solvent distillation. Monomer A3-1 was obtained as colorless oily matter. Amount 62.3 g, yield 48%.

It has been demonstrated that the preparation method of the invention ensures to produce a tertiary ester-containing aromatic vinyl monomer of high quality in high yields as compared with the prior art methods.

Japanese Patent Application No. 2023-185147 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A method for preparing a tertiary ester-containing aromatic vinyl monomer wherein a metal alkoxide is used as a reaction promoter.

2. The method of claim 1 comprising the step of reacting an N-acylimidazole compound with a tertiary alcohol compound using a metal alkoxide as a reaction promoter.

3. The method of claim 2 wherein the N-acylimidazole compound has the formula (A1), the tertiary alcohol compound has the formula (A2), and the tertiary ester-containing aromatic vinyl monomer has the formula (A3): 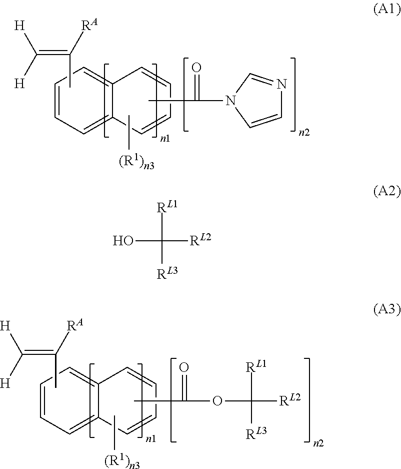
wherein n1 is 0 or 1, n2 is 1 or 2, n3 is an integer of 0 to 6, n2+n3 is from 1 to 5 when n1=0 and n2+n3 is from 1 to 7 when n1=1,
R^{A} is hydrogen, fluorine, methyl or trifluoromethyl,
R¹ is halogen, a C₁-C₂₀ hydrocarbyl group which may contain a heteroatom, or C₁-C₂₀ hydrocarbyloxy group which may contain a heteroatom,
R^{L1}, R^{L2} and R^{L3} are each independently a C₁-C₃₀ hydrocarbyl group, any two of R^{L1}, R^{L2} and R^{L3} may bond together to form a ring with the carbon atom to which they are attached, and —CH₂— in the hydrocarbyl group and the ring may be replaced by —O— or —S—.

4. The method of claim 1 wherein the metal alkoxide is an alkali metal alkoxide or alkaline earth metal alkoxide.

5. The method of claim 4 wherein the metal alkoxide is an alkali metal alkoxide.

6. The method of claim 5 wherein the alkali metal alkoxide has a sodium or potassium cation.

7. The method of claim 5 wherein the alkali metal alkoxide has an alkoxide anion containing a tertiary carbon atom.
